(19) United States
(12) Patent Application Publication
(10) Pub. No.: US 2003/0187432 A1
(43) Pub. Date: Oct. 2, 2003

(54) External fixation system

(76) Inventors: Tab C. Johnson, Ossian, IN (US); Dale G. Davison, Warsaw, IN (US); Tom Ferro, Ft. Wayne, IN (US); Robert C. Broshar, Syracuse, IN (US); George J. Haidukewych, Rochester, MN (US); James B. Carr, Elgin, SC (US); Lon Weiner, Rumson, NJ (US); John Sontich, Novelty, OH (US)

Correspondence Address: Paul J. Maginot, Maginot, Addison & Moore, Bank One Center/Tower, 111 Monument Circle, Suite 3000, Indianapolis, IN, 46204-5115, US

(21) Appl. No.: 10/109,033

(22) Filed: Mar. 28, 2002

(51) Int. Cl. A61B 17/56

(52) U.S. Cl. 606/59

(57) ABSTRACT

A modular external fixation system includes a plurality of modular components, each formed of a non-metallic material. One of the modular components is a modular rod that is provided in a single initial length and then cut to a length appropriate for a particular external fixation procedure. Another modular component is an interlocking element that can be mounted on one end of the modular rod and that includes an interlocking feature for engagement with other modular components. The system can also include a distraction arm that can be mounted at one end of the fixation framework. The arm includes a bar portion configured to be grasped to apply a distraction force along the construct. The arm can be pivotably mounted so that it can be pivoted to a distraction position, or to other positions, including one position in which the arm bears against the foot of an instrumented leg, and another position in which the arm acts to support the entire leg. A pin clamp is provided that has an interlocking feature and an integral spring associated with the feature to prevent interlocking engagement until the pin clamp has been clamped on a bone-engaging fastener. Another pin clamp is provided as a stack of identically configured spacers, with each spacer including a central bore for mounting on a clamping rod, and notches on opposite surfaces of the spacer. The notches of adjacent spacers combine to form a pin bore for receiving and clamping a bone-engaging fastener.

### BACKGROUND OF THE INVENTION

The present invention concerns external fixation systems for the treatment of the skeleton. In particular, the invention relates to a modular fixation system that can be used for the temporary fixation of a variety of bones in the skeleton.

A variety of orthopaedic treatments have been developed for bone fractures and disorders. One such treatment involves the use of an external fixation system. In the typical system, a bone-engaging fastener, such as a screw, nail or pin, is engaged at different positions along a bony structure. The pins project outside the patient and are available for inter-connection using an external support structure or framework. In a typical framework, the bone fasteners are attached to elongated beams or rods using a variety of types of clamps. The framework can provide a mechanism for distraction or compression of a bony structure, as required in the particular circumstance. Once the position of the bone has been fixed, the bone engaging elements can be fixed in position to the external framework.

Most external fixation systems are relatively bulky and include a great number of components, depending upon the bony structure involved in the fixation. The number of components increases greatly where the fixation spans across a joint space. For instance, treatment of an injury to the knee may require attaching bone fasteners to both the femur and tibia, and then linking these attachments together across the knee joint. Obviously, variations in patient anatomy necessitate different sizes and types of external fixators for different patients. In addition, in some instances fixation of other bony structures may be required, such as attachment to the bones of the foot.

There is a continuing need for external fixation systems that exhibit a greater degree of versatility than present systems. Moreover, there is a need for external fixation systems that are universal, meaning that they can be used for a variety of bony structures.

In order to simply the fixation procedure, a desirable external fixation system would minimize the number of loose components that must be connected to form the framework. In most cases, it is also important that the external fixation exhibit a high degree of modularity.

### SUMMARY OF THE INVENTION

In order to address these needs, the present invention contemplates a modular external fixation system that includes a variety of modular components. In one embodiment, each of the modular components is formed of a non-metallic material, such as a plastic. One of the components can include a rod member that is provided in a single initial length and then cut to a length appropriate for a particular external fixation procedure.

Another modular component is an interlocking element that can be mounted on one end of the modular rod and that includes an interlocking feature for engagement with other modular components. The system can also include a distraction arm that can be mounted at one end of the fixation framework. The arm includes a bar portion configured to be grasped to apply a distraction force along the construct. The arm can be pivotably mounted so that it can be pivoted to a distraction position, or to other positions, including one position in which the arm bears against the foot of an instrumented leg, and another position in which the arm acts to support the entire leg.

In one embodiment of the invention, an elongated rod member is provided having opposite ends and defining a post bore in at least one of the opposite ends. A interconnecting element can be provided which includes an engagement surface for engaging other components of the external fixation system. The interconnecting element can be provided in the form of a flange carrying an interlocking feature, such as radial splines. The interconnecting element further includes a post sized to be received within the post bore in the rod member. Means are provided for fixing the post within the post bore. In a preferred embodiment, the means for fixing includes at least one pin bore defined in the rod member and intersecting the post bore, and a bore defined in the post that is alignable with at least one pin bore when the post is within the post bore. A pin can be provided that is sized to extend through the pin bore and the bore defined in the post. Preferably, the pin is press-fit in at least one pin bore. In certain embodiments, the rod member includes a post bore at one end only, or at both ends for carrying two interconnecting elements.

In another aspect of the invention, the modular external fixation system comprises a plurality of modular components including at least one elongated rod member sized for spanning at least a portion of the length of a bone, at least one pin clamp configured for clamping a portion of a bone-engaging fastener, and a rod clamp corresponding to each of said at least one pin clamp and configured for clamping a portion of said elongated rod member. Each of the modular components is formed of a non-metallic material, such as a plastic. Thus, each of the components can be readily molded and can incorporate interlocking features where needed.

The plurality of modular components can further include an intermediate connector including a first body defining an interlocking feature for engaging said interlocking feature of said interlocking element and a second body defining an interlocking feature for engaging another component of the external fixation system. The second body includes a portion variably connectable to the first body. A clamp is provided for fixing the position of the second body relative to the first body, preferably in the form of a C-clamp portion on the first body. The intermediate connector is particularly useful for providing external fixation across a joint, such as the knee. The intermediate connector can be engaged to a modular rod attached to the femur and tibia and can be oriented to account for any off-axis alignment of the two modular rods.

In another aspect of the invention, the plurality of modular components can also include an angle connector including an upper body and a lower body, each defining an interlocking feature. The interlocking feature on the lower body is oriented at a non-parallel angle relative to the interlocking feature on said upper body. Most preferably, this non-parallel angle is a right angle. The angle connector can be used in conjunction with the intermediate connector to provide external fixation across a joint.

In another feature of the invention, an external fixation system is provided for applying a distraction force to a bone. In its simplest form, the system can include an elongated rod sized to span at least between bone-engaging fasteners mounted within the bone, and an arm connectable to the elongated rod. The arm includes a gripping portion disposed beyond the end of the bone that is configured to be grasped to apply a distraction force along the elongated rod. Preferably, the arm is in an L-shape, having a first rod portion configured for connection to the rod, and a second rod portion providing the gripping portion.

The arm can be connectable to the rod at different angular orientations relative to the axis of the rod. Thus, the system can further include a component disposed at one end of the rod defining an interlocking feature and an interlocking feature defined at a first rod portion configured for interlocking engagement with the interlocking feature of the component to permit said different angular orientations. The arm can thus be pivoted to a variety of positions. In one position, the arm is generally aligned with the axis of the rod so that a distraction force can be applied using the arm. In another position, the arm is at substantially right angles to the axis of the rod. In this position, the second rod portion of the arm can bear against and support the foot of an instrumented leg to prevent the foot from drooping. In yet another position, the arm is rotated substantially 180° opposite the “foot droop” position. In this position, the arm can support the entire instrumented leg on another surface.

In yet another aspect of the invention, a pin clamp can be provided comprising a body defining a pin bore sized to receive a portion of a bone-engaging fastener therethrough, together with means for clamping the body on the portion of the bone-engaging fastener within the pin bore. In one embodiment, the body can be in the form of a C-clamp. The body further defines a fastener bore for receiving a clamping fastener therethrough. The body includes a surface defining an interlocking feature thereon, with the fastener bore intersecting the surface. In an important feature of this aspect of the invention, the body includes an integral spring extending from the surface. Preferably, the body and spring are integrally formed in a molding or casting process.

Most preferably, the integral spring is in the form of a helical spring concentrically disposed around the fastener bore. The integral spring helps keep the interlocking feature from engaging an interlocking feature of an adjacent component until a clamping force has been applied between the components.

In yet another feature of the invention, a pin clamp assembly comprises a plurality of pin clamp spacers, each defining a bore therethrough and each defining opposite faces for clamped engagement between adjacent pin clamp spacers. The assembly includes a rod sized to extend through the bore through each of the plurality of pin clamp spacers, so that the spacers can be stacked along the rod when the rod is used as part of an external fixation system. The assembly includes means for clamping the plurality of pin clamp spacers stacked along the length of the rod. In one embodiment, the rod is threaded and includes an end portion defining a shoulder at one end of the rod. A nut can be threaded onto the opposite end of the rod to compress the stack of spacers together.

At least one of the opposite faces of the pin clamp spacers defines a notch configured to receive a portion of a bone-engaging fastener therein when the pin clamp spacers is clamped along the rod. Most preferably, the plurality of pin clamp spacers are identically configured pin clamp spacers, each defining at least one notch on each of the opposite faces of the spacers. When the spacers are stacked on the rod, the notch in adjacent pin clamp spacers defines a combined pin bore sized to receive a portion of a bone-engaging fastener in clamped engagement therein.

It is one object of the present invention to provide a modular fixation system that can be used in a wide range of configurations. Another object is achieved by features that allow the system to provide fixation across joints, such as the knee.

These objects and certain benefits of the invention can be learned from the following written description taken together with the accompanying figures.

### DESCRIPTION OF THE FIGURES

FIG. 1 is an exploded perspective view of certain components of an external fixation system in accordance with one embodiment of the present invention.

FIG. 2 is an enlarged partial cross-sectional view of a rod clamp and pin connector for use with the external fixation system shown in FIG. 1.

FIG. 3 is an enlarged perspective view of an end of a modular rod member for use with the external fixation system shown in FIG. 1.

FIG. 4 is an enlarged perspective view of a limb fixation and distraction component for use with the external fixation system shown in FIG. 1.

FIG. 5 is a front elevational view of the limb component shown in FIG. 4, as depicted in a distraction position.

FIG. 6 is a side elevational view of the system shown in FIGS. 4 and 5, configured in a foot elevation or support position.

FIG. 7 is a perspective view of additional components for use with the external fixation system depicted in FIG. 1.

FIG. 8 is pictorial view of a rod clamp in accordance with a further embodiment of the present invention.

FIG. 9 is a pictorial view of a pin clamp spacer in accordance with an additional embodiment of the present invention.

FIG. 10 is a side perspective view of a pin clamp spacer assembly utilizing the spacer shown in FIG. 9.

### DESCRIPTION OF THE PREFERRED EMBODIMENTS

For the purposes of promoting an understanding of the principles of the invention, reference will now be made to the embodiments illustrated in the drawings and described in the following written specification. It is understood that no limitation to the scope of the invention is thereby intended. It is further understood that the present invention includes any alterations and modifications to the illustrated embodiments and includes further applications of the principles of the invention as would normally occur to one skilled in the art to which this invention pertains.

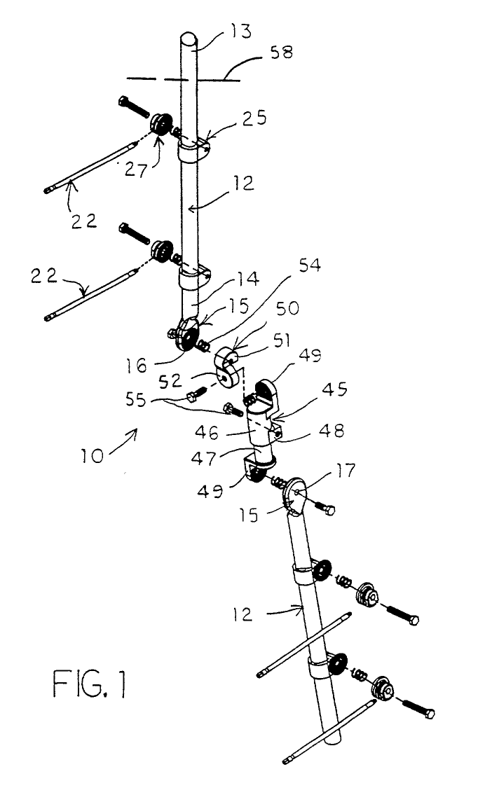

The present invention contemplates an external fixation system, such as the system 10 shown in FIG. 1, that is particularly suited as a temporary fixation system. Moreover, the assembly 10 is particularly well suited to fixation across a joint space. Further, the assembly 10 is modular so that it can be used in a variety of applications for fixation of different skeletal elements.

In a preferred embodiment illustrated in FIG. 1, the fixation system 10 is configured for attachment across a joint, such as the knee joint. For the purposes of the present description, it can be presumed that the system 10 can be mounted to the femur and tibia across the knee joint. The fixation system 10 includes a pair of elongated modular rod member members 12. Each of the rod members can include a free end 13 and a pivot end 14. The pivot end includes a flange element 15 that defines an interlocking feature 16 along with a central bore 17 for receiving a screw, such as a screw 55 as described below. The interlocking feature 16 is preferably in the form of radial splines to allow variable rotational attachment of the modular rod member 12 to another component, including another modular rod member pivot end 14. While radial splines are a preferred form of the interlocking feature 16, other similar features are contemplated by the present invention that allow interlocking of adjacent components to prevent their relative movement in certain degrees of freedom.

In accordance with one feature of the present invention, the modular rod member members 12 can be formed of a readily disposable material. Preferably, the rod is formed of a rigid plastic that is capable of withstanding the stress and strains normally associated with an external fixation system. The plastic material can be a polycarbonate material, carbon fiber or a reinforced plastic that exhibits the requisite strength characteristics. Since the modular rod member 12 is intended to be readily disposable, the material does not need to have significant wear capabilities. Moreover, a plastic material can be easily molded, formed or extruded to a variety of lengths and diameters, and can even include features integrally molded into the rod body. For instance, in one embodiment, the flange element 15 can be integrally formed with the remainder of the modular rod member 12.

One benefit of the modular rod member 12 of the present invention is that it can be provided in a single length and then cut to fit in the operating room. Thus, it is contemplated that the modular rod member, such as the upper rod 12, can be shortened by making a cut at line 58. The cut is preferably made before the rod is fixed to the bone-engaging fasteners. Thus, in a surgical procedure, once the bone fasteners are in place in the patient's bone, the modular rod member 12 can be positioned adjacent to the bone to be instrumented and the appropriate length of the rod marked for cutting. Once the free end 13 of the rod has been cut to size, the rod can then be attached to the bone fasteners.

Looking now to FIG. 3, one feature of the inventive modular rod member 12 is depicted. Specifically, it can be seen that the flange element 15 can be provided as a separate component from the modular rod member 12. The flange element 15 includes a connection post 18 projecting therefrom. The post is sized to be received within post bore 19 formed at the pivot end 14 of the rod 12. The flange element 18 can be locked in position by way of a press pin 21 extending through pin bores 20 in the rod and a corresponding bore 18a in the connection post 18. The press pin 21 can be sized for a press fit within one of the bores 20 or the bore 18a. Alternatively, the pin 21 can be swaged within the pin bore to hold its position.

The pivot end 14 of the modular rod member 12 can include a number of diametrically disposed pin bores 20 dispersed around the circumference of the rod. Thus, the flange element 18 can be oriented at a variety of rotational positions in order to meet the particular external fixation needs. In addition to providing a number of pinning locations, the post bore 19 and the post 18 can include complementary noncircular profiles. In this way relative rotation between the flange element 15 and the rod 12 can be prevented. For example, the bore and post can have complementary hexagonal shapes so that the flange element 15 can be rotated to six different positions relative to the rod.

To further enhance the modularity of the rod, a post bore 19 can be formed at the opposite ends of the rod. Thus, the flange element 15 can be disposed at either end or at both ends, again as required by the particular external fixation needs. Alternatively, the post bore 19 can extend entirely through the length of the rod 12, so that the rod is in the form of a tube. In this way, the post bore 19 will remain even if the free end 13 of the rod is cut to a particular length along the cut line 58, as described above. When the external fixation system 10 is constructed, the axial extending bore can be filled, either with a stiffing rod, or with a hardenable stiffing material, in order to improve the stiffness properties of the rod.

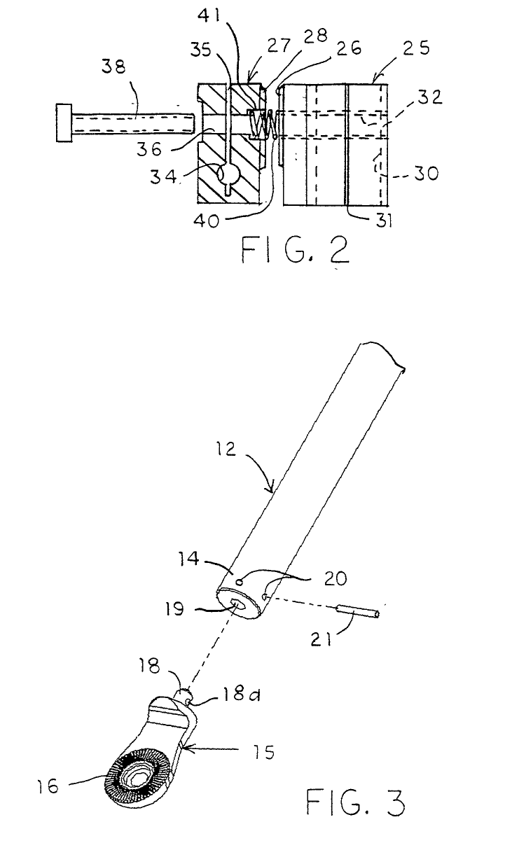

Referring back to FIG. 1, it can be seen that the fixation pins 22 are connected to the rod 12 by way of a rod clamp 25 and a pin clamp 27. Details of these two clamps are depicted in FIG. 2. In particular, the rod clamp 25 can include an interlocking feature 26 on one face, which feature can be the same as the feature 16 discussed above. The rod clamp 25 defines a rod bore 30 through which the rod 12 extends. In addition, the clamp can include a clamping slot 31 which intersects the rod bore 30 and allows the rod clamp to operate in the form of a C-clamp. Preferably, the rod bore 30 has a diameter when the clamping slot 31 is closed that is slightly smaller than the diameter of the rod 12. Thus, the clamp 25 can exert a clamping force on the rod by closing the clamping slot 31.

The pin clamp 27 also includes an interlocking feature 28 on a surface that faces the interlocking feature 26 of the rod clamp 25. Again, this interlocking feature can be similar to that described above provided that it allows the rod clamp 25 and the pin clamp 27 to be fixed at variable rotational positions relative to each other. The pin clamp 27 defines a pin bore 34 which receives a fixation pin 22. The clamp also defines a clamping slot 35 that operates in the same manner of the clamping slot 31 of the rod clamp 25.

In order to fix the components together and to force the respective clamping slots 31 and 35 closed, a clamping screw 38 is provided. The screw 38 extends through a bore 36 in the pin clamp 27 and engages a threaded bore 32 in the rod clamp 25. Thus, as the screw is tightened within the threaded bore 32, it exerts pressure first against the pin clamp 27 to close the clamping slot 35, and then against the rod clamp 25 to close its clamping slot 31. In this manner, both clamps are firmly affixed to their respective rod 12 or fixation pin 22. Moreover, when the screw 38 is tightened, the interlocking features 26 and 28 engage to prevent relative rotation between the two components, and ultimately to prevent relative rotation of the rod 12 relative to the bone-engaging fastener 22.

When the external fixation system 10 is initially constructed, a rod clamp/pin clamp construct is slid onto the rod. With this construct, the screw 38 is loosely engaged within the threaded bore 32 so that the two clamps 25 and 27 are free to rotate relative to each other. In order to maintain this freedom of rotation, a spring 40 can be disposed between the two clamps 25 and 27, and particularly between their respective interlocking features 26 and 28. The spring 40 is preferably a helical spring that is concentrically disposed with the threaded bore 32 and the screw bore 36. Thus, the clamping screw 38 extends through the spring 40 to hold the spring in position. The spring 40 is calibrated to produce a slight separation force between the rod clamp 25 and the pin clamp 27 to keep the two interlocking features 26, 28 apart.

Preferably, the spring 40 is partially situated within a spring bore 41 defined in the face of the clamp bearing the interlocking feature 28. The bore 41 is illustrated in FIG. 2 as being defined in the pin clamp 27. However, the bore can be equally well defined in the rod clamp 25, or in both the rod clamp and the pin clamp. At any rate, the bore provides a space for the compressed spring 40 to reside once the clamping screw 38 is fully engaged within the threaded bore 32.

As thus far described, the external fixation system 10 of the present invention includes a modular rod member 12 with a modular flange element 15 that provides a means for interlocking engagement with other components of the system. For instance, two modular rod members 12 can be engaged to each other by way of corresponding flange elements 15. The rod clamp 25 and the pin clamp 27 allow attachment of a fixation pin 22 to a modular rod member 12 at whatever axial or rotational orientation is necessary.

Thus, in a typical fixation installation, the fixation pins 22 will be engaged within the particular bony structure so that the pins extend from the patient. The modular rod member can than be positioned adjacent to the exposed ends of the pins, with rod clamp 25 and pin clamp 27 constructs pre-threaded on the rod. The multiple degrees of freedom of movement of the two clamps 25 and 27 allows the pin bore 34 to be readily aligned with the exposed portion of a corresponding fixation pin 22. In this respect, fixation system 10 can be used to provide distraction of a single bone, such as a long bone. The fixation pins can be moved apart by an appropriate distraction device while they are loosely connected to the modular rod member 12. Once the distraction of the bony structure is complete, the rod clamps 25 and pin clamps 27 can be tightened to fix the position of the fixation pins 22 on the rod 12.

One beneficial feature of the external fixation system 10 of the present invention is that it can be utilized for fixation across a joint, such as a knee joint. Thus, in another aspect of the inventive fixation system 10, an intermediate connector 45 can be provided. The connector 45 includes a main body 46 that integrates with a rotating/telescoping member 47. A clamp 48 is defined on the main body 46 that can be tightened with a screw 55 to fix the relative position of the rotating/telescoping member 47 with respect to the main body 46. Both bodies include an attachment flange 49 that can incorporate an interlocking feature similar to the features described above. Thus, the intermediate connector 45 can be connected between flange elements 15 on different modular rod members 12 to permit adjustment of relative angular position between the rods, as well as distance between the rods.

To further enhance the modularity and in particular the angular position between two rods 12, the fixation system 10 can incorporate a right angle connector 50. The right angle connector 50 includes an upper body 51 and a lower body 52 that are disposed at a non-parallel angle relative to each other, and most preferably at a right angle. Both bodies 51, 52 can define interlocking features on their exposed faces, again as described above.

As shown in FIG. 1, the right angle connector 50 can be disposed between the flange element 15 of modular rod member 12 and the attachment flange 49 of intermediate connector 45. In addition to providing an immediate right angle change in orientation, the connector 50 allows the rod 12 to assume variable angular or rotational positions relative to the connector, and likewise with respect to the intermediate connector 45. The right angle connector 50 can be implemented anywhere that there is a flange component having an interlocking feature. It can thus be appreciated that the external fixation system 10 allows for full modular construction so that any of the components 12, 45 and 50 can be combined as necessary to accommodate particular fixation needs. The provision of the intermediate connector 45 and the right angle connector 50 accounts for installations where the modular rod members 12 are not aligned along a common axis or parallel to each other. For instance, in some installations it may be necessary to accommodate a bend at a joint. The intermediate connector 45 and the right angle connector 50 facilitate accommodating this bend.

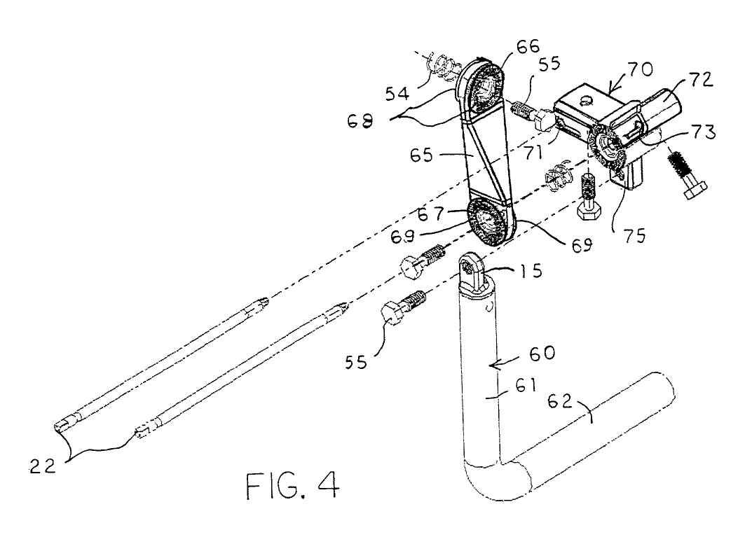

In the embodiment illustrated in FIG. 1, the external fixation system 10 includes two long modular rod members 12 that are particularly well suited for engagement to the femur and tibia across the knee joint. Another modular element that can become part of the fixation system shown in FIG. 1 is depicted in FIG. 4. In particular, this modular element contemplates a distraction arm 60 that can be used to provide a distraction force to an installation before all of the various clamps are tightened. Thus, the distraction arm 60 preferably includes a first bar 61 and second bar 62 projecting from the first bar. Most preferably, the distraction arm 60 forms an L-shape. The second bar 62 thus provides a gripping surface so that the distraction arm 60 can be pulled by the orthopaedic surgeon. As the distraction arm 60 is pulled, it can apply a distraction force to a portion of bone to which a fixation pin 22 has been attached.

In a simple installation, the distraction arm 60 can be engaged to a flange element 15 of a modular rod member 12. In order to apply a distraction force, a lowermost fixation pin 22 can be affixed or clamped to the modular rod member 12 while the uppermost fixation pin can be loosely connected to the rod without tightening the associated rod clamp and the pin clamp. As the distraction arm 60 is pulled by the orthopaedic surgeon, the distraction force is transmitted to the lower portion of the bone structure through the lowermost fixation pin. Once the distraction is complete, the uppermost fixation pin can be clamped onto the modular rod member 12.

In the particular embodiment illustrated in FIG. 4, the distraction arm 60 is connected to the external fixation system 10 by way of a right angle connector 65. The connector 65 includes opposite first and second ends 66 and 67 that are oriented at a non-parallel angle relative to each other, again preferably a right angle. The two ends 66, 67 define interlocking features 68, 69 at opposite faces of the ends. Thus, the right angle connector 65 can be placed in any particular orientation to still provide the variable rotational connections capabilities.

Further in the illustrated embodiment of FIG. 4, a pin block 70 is provided that accommodates multiple fixation pins at a particular level along the construct. In the illustrated embodiment two such pins 22 can be carried by the pin block 70, such as may be used for fixation to the foot of a patient. The pin block 70 can include a first pin bore 71 and a second pin bore 73 formed in an angled extension 72. As with the other interlocking components, a spring 54 and clamping screw 55 can be used to clamp the various components together. Of course, the spring 54 is only provided where interlocking features are involved so that the features maybe kept apart while the relative angular orientations are fixed.

In order to accommodate the distraction arm 60, the pin block 70 can include an arm connection element 75 projecting from the underside of the block. A clamping screw 55 can fix the distraction arm 60 to the arm connection element 75. An interlocking feature can be incorporated between the two components. For instance, in the preferred embodiment, the distraction arm 60 can include a flange element 15 of the construction shown in FIG. 3. The arm connection element 75 thus allows the distraction arm 60 to be connected to the construct by way of the pin block 70. The advantage of this orientation is that the pin block 70 permits multiple fixation points to the skeletal structure, which can more easily absorb the stress of a distraction force being applied to the bony structure.

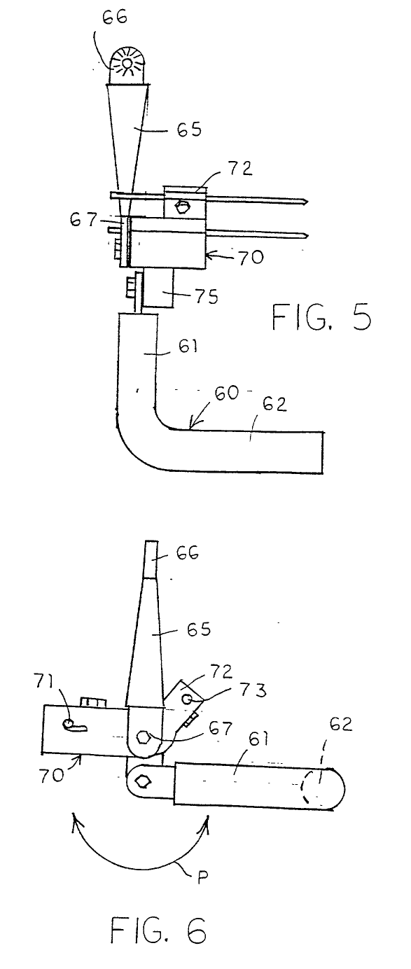

A further benefit of the distraction arm 60 of the present invention can be seen by comparing FIGS. 5 and 6. As shown in FIG. 5, the distraction arm 60 is oriented in its distraction position. Specifically, the second bar 62 projects below the external fixation construct so that it can be readily grasped by the orthopaedic surgeon to apply a distraction force.

However, as shown in FIG. 6, the distraction arm 60 can be rotated or pivoted in the direction of the arrows P. When the pin block 70 is attached to a bone of the foot, the distraction arm 60 can be pivoted to the position shown in FIG. 6 so that the second bar 62 provides a resting support for the length of the patient's foot. Thus, the distraction arm 60 can be used to prevent droop of the foot when the foot and lower leg are instrumented.

Alternatively, the distraction arms 60 can be rotated to a position generally 180° opposite that shown in FIG. 6. In this position, the distraction arm 60, in particular the second bar 62, serves as a support for supporting the foot and lower leg on another surface. It can thus be appreciated that the distraction arm 60 continues the modularity aspect of the external fixation system 10 of the present invention. The distraction arm 60 also provides additional utility not available in prior fixation systems.

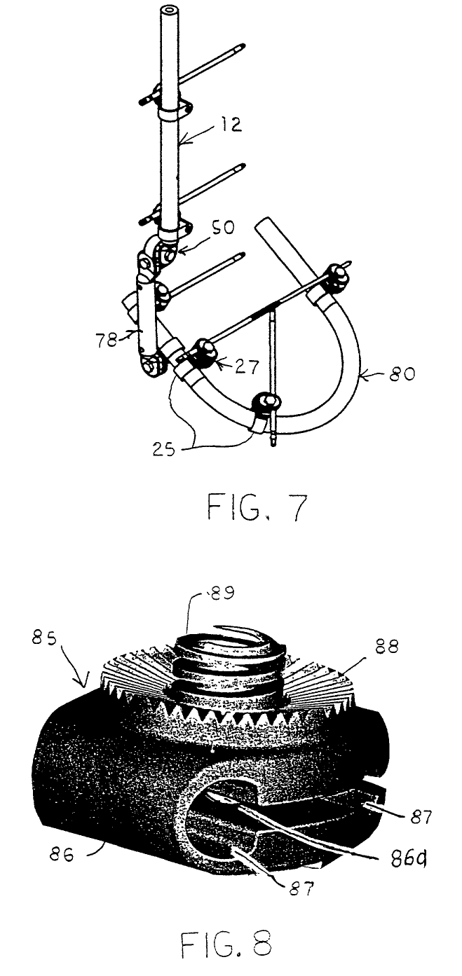

The modularity of the external fixation system 10 of the present invention is further enhanced by the addition of a short rod 78 and a U-rod 80, as depicted in FIG. 7. In the installation shown in this figure, a right angle connector 50 can connect a modular rod member 12 to the short rod 78. The short rod can then be connected to the U-rod 80 using the similar rod clamp 25 and pin clamp 27 construction as described above. The U-rod 80 can be used in a conventional manner to support a member of fixation pins 22 engaged to a bone. It can be seen in the arrangement shown in FIG. 7 that the right angle connector 50 provides a means for offsetting the alignment of the short rod 78 relative to the modular rod member 12. This offset can help accommodate the U-rod 80 so that the rod can be situated immediately adjacent the patient. It can be further appreciated that the tubular or cylindrical U-rod 80 allows of infinitely variable positioning of a fixation pin relative to the rod.

In the embodiments illustrated thus far, the pin clamp 27 has been utilized. This pin clamp can be replaced by an alternative inventive pin clamp 85 show in FIG. 8. The pin clamp 85 can include a C-shaped body 86 that defines a pair of pin bores 87 for engaging two fixation pins 22. As with the prior pin clamp 27, the clamp 85 can define a bore 86a for receiving a clamping screw 38 therethrough. Likewise, the pin clamp 85 can include an interlocking feature 88, which in the illustrated embodiment constitutes an array of radial splines. Preferably, the interlocking feature is integrally formed with the body 86.

In accordance with the pin clamp 85 of the present invention, an integral spring 89 can be defined on the C-shaped body 86. The integral spring 89 can thus replace the spring 40 described above. The integral spring 89 serves the same function—i.e., to keep the interlocking components separated until the installation is finally clamped together. In the illustrated embodiment, the pin clamp 85 is preferably formed from an injection molded plastic. Thus, the integral spring 89 can be formed as part of the clamp body 86. This construction provides the advantage of eliminating an extra part, namely the spring 40. Moreover, the integral spring 89 will always be maintained in alignment with the bore 86a for receiving the clamping screw 38.

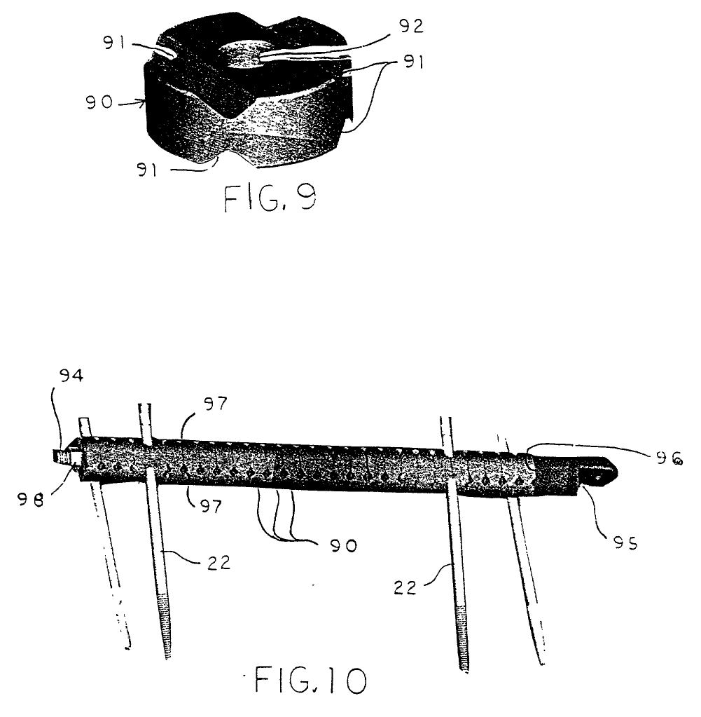

In a further embodiment of the invention, a pin clamp spacer 90 is provided as shown in FIG. 9, that can be stacked together as shown in FIG. 10. The pin clamp spacer 90 defines a number of pin notches 91. In the preferred embodiment, a pair of notches is defined on each of the opposite faces of the spacer 90. The spacer also defines a rod bore 92 through the center of the spacer.

As shown in FIG. 10, the rod bore 92 accommodates a threaded rod 94 extending therethrough. Thus, a plurality of the spacers 90 can be stacked along a single threaded rod 94. Preferably, the rod 94 includes a connector end 95 that is adapted for connecting to other components of the modular external fixation system 10, as described above. The connector end 95 defines a shoulder 96 against which a first spacer 90 in the stack can bear. As can be appreciated from the figure, the pin notches 91 in adjacent spacers 90 define a combined pin bore 97. The combined pin bore 97 of spacers that are in direct contact defines an opening that is smaller than the diameter of a fixation pin 22. Thus, when a clamping force is provided to the adjacent spacers, they exert a significant clamping force on the corresponding fixation pin.

In order to achieve this clamping, the threaded rod 94 is provided with a clamping nut 98 at the end of the rod opposite the connector end. Thus, as shown in FIG. 10, a plurality of the spacers can be stacked together along the threaded road 94. The spacers 90 are at first loosely situated on the rod so that fixation pins can be readily disposed within combined pin bores 97. As shown in FIG. 10, the pins 22 can be connected on opposite sides of the rod 94 due to the availability of pin notches 91 on the opposite faces of the spacers 90. It can be further appreciated that the combined pin bores 97 need not be entirely aligned along the length of the threaded rod. Thus, the spacers 90 can be rotated to an appropriate position to accept a particular orientation of fixation pin 22. Once all of the fixation pins have been engage within a corresponding combined pin bore 97, the clamping nut 98 can be tightened. The nut exerts a clamping force along the entire length of the threaded rod 94 to clamp all of the spacers together.

In a preferred embodiment each spacer is uniformly sized and shaped. In alternative embodiments, spacers having longer or shorter lengths can be provided. For instance, as shown in FIG. 10, the central portion of the threaded rod 94 is not engaged to any fixation pin 22. Thus, a single long spacer can be provided for spanning that intermediate portion. As a further alternative, the opposite faces of the spacers 90 can be provided with an interlocking feature, of the sort described above. This interlocking feature will help the spacers resist relative rotation along the axis of the threaded rod 94. The spacers can even incorporate the integral spring 89 shown in connection with the clamp 85.

It can be appreciated that the pin clamp spacer 95 further enhances the overall modularity of the fixation system 10 of the present invention. Specifically, the spacers provide a very rigid construct while permitting attachment of fixation pins at any location along the length of the rod.

While the invention has been illustrated and described in detail in the drawings and foregoing description, the same should be considered as illustrative and not restrictive in character. It is understood that only the preferred embodiments have been presented and that all changes, modifications and further applications that come within the spirit of the invention are desired to be protected.

For instance, one embodiment of the invention contemplates a modular rod member 12 that is formed of a plastic material. The same material can be used to form the other components of the inventive fixation system 10. Thus, the rod clamps 25, pin clamps 27 and 85, right angle connectors 50 and 65, intermediate connector 45, distraction arm 60, pin block 70, and pin clamp spacers 90 can all be formed of the same plastic material in an injection molding process.

What is claimed is:

1. A modular rod for use with an external fixation system comprising:
an elongated rod member having opposite ends and defining a post bore in at least one of the opposite ends;
a flange element including;
an engagement surface for engaging other components of the external fixation system; and
a post sized to be received within said post bore in said rod member; and
means for fixing said post within said post bore.

2. The modular rod member according to claim 1, wherein said engagement surface includes radial splines.

3. The modular rod member according to claim 1, wherein a post bore is defined in both of said opposite ends.

4. The modular rod member according to claim 1, wherein said means for fixing includes:
at least one pin bore defined in said rod member and intersecting said post bore;
a bore defined in said post and alignable with said at least one pin bore when said post is within said post bore; and
a pin sized to extend through said at least one pin bore and said bore defined in said post.

5. A modular external fixation system comprising:
a plurality of modular components including;
at least one elongated rod member sized for spanning at least a portion of the length of a bone;
at least one pin clamp configured for clamping a portion of a bone-engaging fastener; and
a rod clamp corresponding to each of said at least one pin clamp and configured for clamping a portion of said elongated rod member,
wherein each of said plurality of modular components is formed of a non-metallic material.

6. The modular external fixation system according to claim 5, wherein said non-metallic material is a plastic.

7. The modular external fixation system according to claim 5, wherein said plurality of modular components includes an interlocking element associated with said elongated rod member and including an interlocking feature for engaging another component of the external fixation system.

8. The modular external fixation system according to claim 7, wherein said plurality of modular components includes an intermediate connector including:
a first body defining an interlocking feature for engaging said interlocking feature of said interlocking element;
a second body defining an interlocking feature for engaging another component of the external fixation system and including a portion variably connectable to said first body; and
a clamp for fixing the position of said second body relative to said first body.

9. The modular external fixation system according to claim 7, wherein said plurality of modular components includes an angle connector including an upper body defining an interlocking feature and a lower body defining an interlocking feature oriented at a non-parallel angle relative to said interlocking feature on said upper body.

10. The modular fixation system according to claim 9, wherein said non-parallel angle is a right angle.

11. A method for engaging the external fixation system of claim 5 to a bone of a patient comprising the steps of:
fixing at least two bone-engaging fasteners into the bone of the patient;
providing said elongated rod member in a first length;
sizing the rod member relative to the length of the bone; and
cutting said elongated rod member to a second length less than said first length according to said sizing step.

12. An external fixation system for providing a distraction force to a bone, comprising:
an elongated rod sized to span at least between bone-engaging fasteners mounted within the bone; and
an arm connectable to said elongated rod and having a gripping portion disposed beyond the end of the bone configured to be grasped to apply a distraction force along said elongated rod.

13. The external fixation system according to claim 12, wherein said arm is connectable to said rod at different angular orientations relative to the axis of said rod.

14. The external fixation system according to claim 13, further comprising:
a component disposed at one end of said rod and defining an interlocking feature; and
a first end of said arm defining an interlocking feature configured for interlocking engagement with the interlocking feature of said component to permit said different angular orientations.

15. The external fixation system according to claim 14, wherein said interlocking features of said component and said arm are configured to permit orientation of said arm at a first position in which said arm is at substantially right angles to the axis of said rod, and a second position substantially 180° opposite said first position.

16. The external fixation system according to claim 12, wherein said arm is L-shaped.

17. A pin clamp comprising:
a body defining a pin bore sized to receive a portion of a bone-engaging fastener therethrough;
means for clamping said body on the portion of the bone-engaging fastener within said pin bore;
a fastener bore extending through said body for receiving a clamping fastener therethrough;
said body including a surface defining an interlocking feature thereon, said fastener bore intersecting said surface; and
an integral spring extending from said surface.

18. The pin clamp according to claim 17, wherein said integral spring is in the form of a helical spring concentrically disposed around said fastener bore.

19. A pin clamp assembly comprising:
a plurality of pin clamp spacers, each defining a bore therethrough and each defining opposite faces for clamped engagement between adjacent pin clamp spacers;
a rod sized to extend through said bore through each of said plurality of pin clamp spacers;
means for clamping said plurality of pin clamp spacers stacked along the length of said rod; and
at least one of said opposite faces of at least one of said plurality of pin clamp spacers defining a notch configured to receive a portion of a bone-engaging fastener therein when said plurality of pin clamp spacers are clamped along said rod.

20. The pin clamp assembly according to claim 19, wherein said plurality of pin clamp spacers includes a plurality of identically configured pin clamp spacers, each defining at least one notch on each of said opposite faces, said notch in adjacent pin clamp spacers defining a combined pin bore sized to receive a portion of a bone-engaging fastener in clamped engagement therein.
